(19) United States
(12) Patent Application Publication
(10) Pub. No.: US 2020/0323939 A1
(43) Pub. Date: Oct. 15, 2020

(54) Method of Extracting Flavonoid from Chrysanthemum Morifolium and Method of Manufacturing Medicine Composition with Effect of Alleviating Dry Eye Syndrome

(71) Applicant: A. T. P. CO., LTD., Taipei (TW)

(72) Inventor: Hsi-Tai Sun, Taipei City (TW)

(21) Appl. No.: 16/844,989

(22) Filed: Apr. 9, 2020

(30) Foreign Application Priority Data
Apr. 9, 2019 (TW) 108112381

(51) Int. Cl. A61K 36/287; A61P 27/04

(52) U.S. Cl. CPC A61K 36/287; A61P 27/04

(57) ABSTRACT

The present invention provides a method of extracting flavonoid from C. morifolium. First, a dried plant material derived from C. morifolium is provided. The dried plant material can include only the leaves and the stems, or can include the stems, the leaves and the flowers. Next, an extraction step is performed with an extractant to obtain an extraction solid and an extraction liquid, wherein the extraction liquid comprises a flavonoid compound with a concentration of 8˜22 mg/g of the dried plant material. The present invention further provides a method of manufacturing a medicine composition with effect of alleviating dry eye syndrome.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention is related to a method of extracting flavonoids from Chrysanthemum morifolium and a method of manufacturing a medicine composition with effect of alleviating dry eye syndrome, and more particularly, is related to a method of extracting flavonoids from Chrysanthemum morifolium and a method of manufacturing a medicine composition with effect of alleviating dry eye syndrome by using a specific part of Chrysanthemum morifolium.

### Description of Related Arts

Chrysanthemum morifolium, which is also known as Juhua, can be taken as medicinal material, beverage, and is also commonly used for gardening. The flower of Chrysanthemum morifolium (hereinafter “C. morifolium”) contains volatile oil, flavonoids, polyphenols, inulin, purine, choline, stachydrine, vitamin A, vitamin B and amino acids. The studies of Chinese and Western medicines suggest that C. morifolium can dispel cold, clear heat, suppress hyperactive liver, improve eyesight, refresh body, promote urination, and calm the nerve system. Bathing with chrysanthemum in a therapeutic bath helps soak aches and pain away and therefore refreshes the body, supports skin health and promotes detoxifications. At the same time, it is said that drinking chrysanthemum tea helps to enhance capillary resistance and inhibit capillary continuity, thereto bring anti-inflammatory effect.

The flavonoid compounds are featured by a 2-phenyl-1-benzopyran-4-one skeleton, wherein the basic core is 2-phenylchromogen ketone compounds, now referring to a series of compounds in which two benzene rings with phenolic hydroxyl groups are connected to each other through a central three carbon atom. They are usually from fruits, vegetables, seeds or plant roots. Although flavonoid compounds are not vitamins, they have vitamin-like nutritional functions such as antioxidant, anti-inflammatory, or tumor inhibitory effects. Consequently, they are sometimes called “vitamin P” and are considered more and more important for human health.

At present, the extraction method of C. morifolium reported in the literatures mainly include: water decoction, acid-base extraction, ethanol reflux method, etc., in which C. morifolium is extracted by methods with limited yield and high cost.

### SUMMARY OF THE PRESENT INVENTION

The present invention therefore proposes a method of extracting flavonoid compounds from C. morifolium, which shows higher extraction efficiency and is suitable for industrial mass production. This preparation method can also be used to manufacture a medicine composition with effect of alleviating dry eye syndrome.

According to one embodiment, the present invention provides a method of extracting flavonoid from C. morifolium. First, a dried plant material derived from C. morifolium is provided. Next, an extraction step is performed with an extractant to obtain an extraction solid and an extraction liquid, wherein the extraction liquid comprises flavonoids with a concentration of 8˜22 mg/g of the dried plant material.

According to another embodiment, the present invention further provides a method of manufacturing a medicine composition with effect of alleviating dry eye syndrome. First, a dried plant material derived from C. morifolium is provided, Then, an extraction step is performed with an extractant to obtain an extraction solid and an extraction liquid, wherein the extraction liquid comprises flavonoids with a concentration of 8˜22 mg/g of dried plant material.

Unlike conventional techniques with uses the flowers of C. morifolium as the main extraction object, the present invention utilizes single parts such as the stems or the leaves or multi parts (for example, the stems/leaves, or the stems/leaves/ flowers) as the main extraction object. By utilizing novel extraction steps, the extraction efficiency can be upgraded comparing to conventional arts.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows the chromatogram of the aqueous phase of the extracts of flowers, leaves and stems of C. morifolium extracted by conventional arts after analyzing with high performance liquid chromatography according to the comparative embodiment, wherein the ratios represented in the figure indicate the dilute ratio of each respective extracts.

FIG. 2 shows the chromatogram of the organic phase of the extracts of flowers, leaves and stems of C. morifolium extracted by conventional arts after analyzing with high performance liquid chromatography according to the comparative embodiment, wherein the ratios represented in the figure indicate the dilute ratio of each respective extracts.

FIG. 3 shows the chromatogram of the organic phase of the extracts of leaves and stems of C. morifolium by the method of the present invention after analyzing with high performance liquid chromatography according to the present embodiment.

FIG. 4 is a comparison chart of the quality of tear fluid after irradiating UV to mice and then feeding the mice with the extracts from C. morifolium.

FIG. 5 shows degrees of cornea damage after irradiating UV to mice after feeding the mice with the extracts from C. morifolium, and the four sub-pictures respectively represent cornea opacity, cornea smoothness, corneal topography, and Lissamine green stain examination.

### DETAILED DESCRIPTION OF THE PREFERRED EMBODIMENT

To provide a better understanding of the presented invention, preferred embodiments will be described in detail. The preferred embodiments of the present invention are illustrated in the accompanying drawings with numbered elements.

The present invention is directed to an extraction method of using C. morifolium as the main extraction object for extracting flavonoid compounds. Unlike most conventional techniques with using the flowers of C. morifolium as the main extraction object, the present invention is featured by using single parts such as the stems or the leaves or multiple parts of C. morifolium (for example, the stems/leaves, or the stems/leaves/flowers) as the main extraction object. Moreover, in one preferred embodiment, the extraction is carried out with a specific proportion of the stems and the leaves thereto simplify the extraction steps, being suitable for commercial mass production.

The extraction method of the present invention will be described in the following context. First, C. morifolium plants are harvested and collected. The harvest plant of C. morifolium may refer to the dry product after cultivated and isolated from the environment and then being dried in a drying step. The drying step may be performed, for example, by exposing the plants to the sun, or being subjected to hot air drying and other similar machines. It is noted that, the present invention is not limited to use single parts of the plant as the extraction object (such as using only flowers, petals or buds), instead, the present invention can use single parts (the stems or the leaves) but also can use multiple parts of the dried plant (e.g., use the stems and the leaves, or use the stems, the leaves and the flowers). In one embodiment, since the harvest plant of C. morifolium may contain the stems or the leaves or at least the stems and the leaves of the dried plant, the harvest season may not be limited to traditional bud differentiation season (e.g., October to December), but may be any appropriate time after planting. Accordingly, it is more suitable in industrial mass production.

For extracting the stems, the leaves and the flowers, please proceed the next step described in the next paragraph. For extracting only the stems and the leaves, a selective flower removing step is performed for the harvest product of C. morifolium, so that the harvest product preferably has only the stems and/or the leaves. The flower removing step refers to removing the flower part of C. morifolium, e.g. petal, flower, as well as the calyx portion preferably. This flower removing step can be taken manual or by using sorting machine. In addition, this flower removing step may also be carried out with the combination of the previous step, that is, when harvesting the plant, the flower parts of the plant can also be removed simultaneously. In another embodiment of the present invention, a root removing step may also be included. Similarly, the root removing step may be performed together or separately with the flower removing step, or may be performed with the foregoing harvesting step simultaneously or separately. After performing the aforementioned flower and/or root removing step, a dried plant material containing stems and/or leaves can therefore be obtained. In one embodiment of the present invention, the stem-to-leaf weight ratio of the dried plant material of C. morifolium may be, for example, 6:4 (3:2) to 8:2 (4:1), optimally 7:3, so as to achieve the best extraction result.

Next, an extraction step is performed. Unlike conventional arts that use water and organic solvents and needs to repeat the extraction step for several times, another salient feature of the present invention is to use a special extractant that can simplify the extraction steps. In the present invention, the extractant is an organic solvent having a proper solubility for the flavonoids. In one embodiment, the extractant is 40%-60% alcohol, preferably 50% alcohol, which is advantageous for less burden to human body and more reliability of food safety in the subsequent steps. Another feature of the present invention is that this step only requires less than four extraction repeats but can yield to an equal or even better extraction result than in conventional arts. In the present invention, the extraction repeats may be two or three. In comparison with conventional arts that requires at least four extraction repeats, the present invention is time saving and brings to higher extraction efficiency. In one embodiment, the extraction condition is at approximately 60° C., and each step is executed for 30 minutes.

After the above extraction steps, an extraction solid and an extraction liquid can be obtained, wherein the extraction liquid has a desired concentration of flavonoids. In the present invention, it is featured that the extraction step can be carried out with very few of repeats to get a desired extraction results while in comparison with conventional method that requires several repeats or needs additional separation steps such column chromatography. Subsequently, the extraction solids can be separated from the extraction liquid. In one embodiment, the extraction liquid may be subjected to a further concentration step to concentrate under reduced pressure to a predetermined volume, i.e., to a volume of ¼, thereto get a refined extract. In one embodiment, the total flavonoid extract concentration in the refined extracts is 8˜22 mg/g of dry plant material.

The extraction liquid or refined extraction liquid obtained by the above method has a high content of total flavonoids, and has the effects of anti-oxidation, anti-inflammatory, and improvement of cardiovascular diseases. In one embodiment of the present invention, the method can be used to prepare a medicinal composition for alleviating dry eye syndrome, and is proven to have good effect comparing to artificial tears.

The detailed description of the above extraction and manufacturing method of the present invention will now be described with reliable effects as follows.

### COMPARATIVE EXAMPLE

Objective: To evaluate the similarities and differences of total flavonoids in extracts of chrysanthemum flowers, stems and leaves in aqueous and organic extracts.

C. morifolium source: Tongluo Township, Miaoli County, Taiwan (R.O.C.)

Extraction conditions: (1) The flowers, stems and leaves of fresh C. morifolium were dried by cold air drying. (2) The dried plant was extracted by using 100% water which is 10 times weight of the dried plant at 60° C., for twice (each for 30 minutes). Then, the extracted solid and liquid are separated. (3) The liquid was concentrated under reduced pressure to a volume of ¼ to obtain an aqueous phase extract. (4) The solid was extracted twice with a solvent (95% edible alcohol) at a constant temperature of 60° C. for twice (30 minutes each time). (5) The liquid was concentrated under reduced pressure to a volume of ¼ to obtain an organic phase extract.

Qualitative evaluation method: Chromatographic analysis is used to respectively analyze the aqueous phase extraction and organic phase extraction at 415 nm with the use of chromatograph equipped with Waters 600E multisolvent delivery system, Waters 2487 UV detector, Waters column heater, CNW C18 analysis column (4.6 mm*250 mm, 5 μm) and QChrom-1S chromatography data processing system. Analysis conditions: The column temperature was set at 30° C., and the column was delivered with an injection volume of 20 μL and a flow rate of 1 mL/min, wherein the mobile phase was prepared by gradient mixing A=Acetonitrile and B=H₂O. The wavelength of the detector was set at 415 nm. The conditions for analyzing aqueous phase are shown in Table 1, and the conditions for analyzing orgnaic phase are shown in Table 2.

TABLE 1

| Time (min) | A | B |
|---|---|---|
| 0 | 100 | 0 |
| 30 | 0 | 100 |
| 35 | 100 | 0 |

TABLE 2

| Time (min) | A | B |
|---|---|---|
| 0 | 0 | 100 |
| 35 | 100 | 0 |
| 40 | 0 | 100 |

Quantitative evaluation method: The spectrophotometer uses colorimetry and quercetin as standards to quantify the total flavonoid content. The total flavonoid content in the aqueous and organic phases was calculated separately.

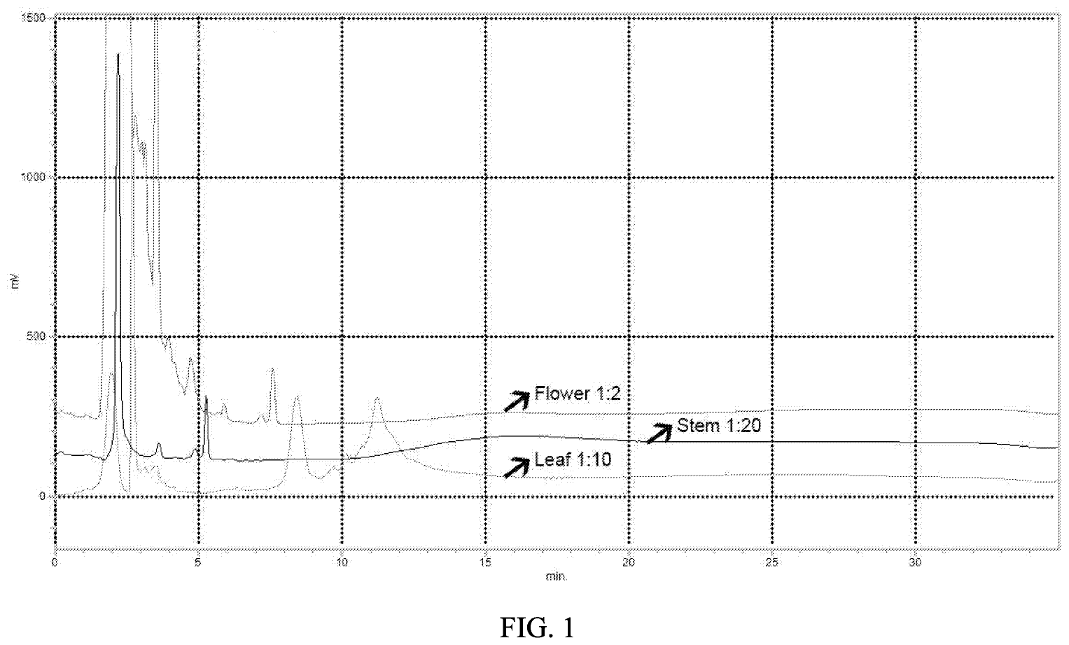

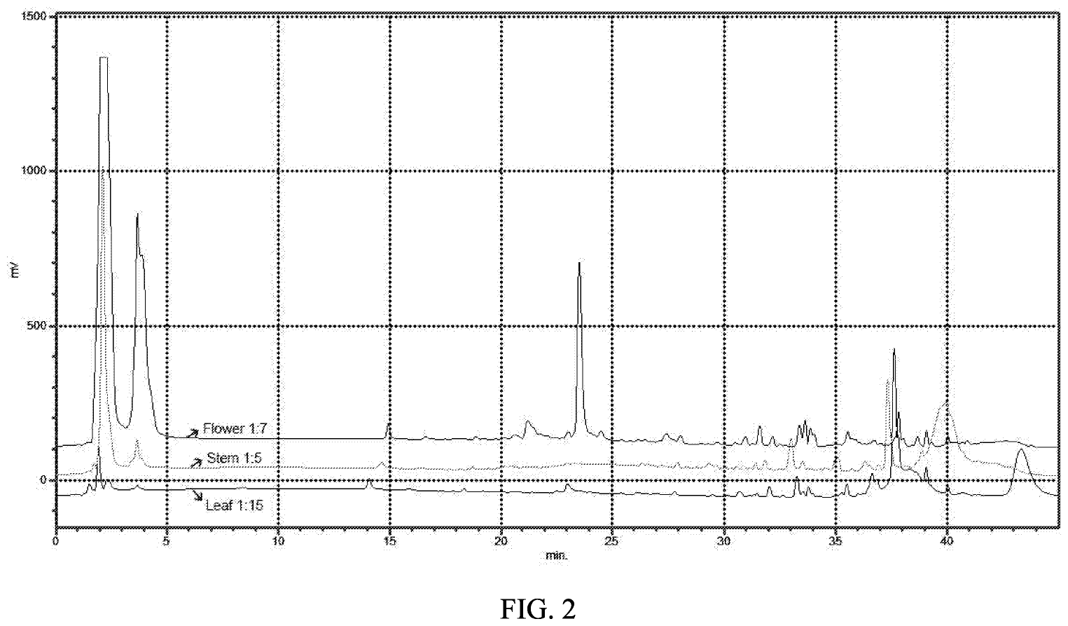

Qualitative results: The aqueous phase is shown in FIG. 1, and the organic phase is shown in FIG. 2. As shown in FIG. 1 and FIG. 2, the contents in aqueous phase of the stems, the leaves and the flowers of C. morifolium are similar to each other, but there is still some difference between the stems, the leaves to the flower portion. In the organic phase, the stems and the leaves exhibit similar fingerprint but are different compared to the flower portion.

Quantitative results: shown in Table 3.

TABLE 3

| | Flavonoid |
|---|---|
| Portion | (mg/g of dried plant) |
| Flowers | 2.9 |
| Stems | 14.1 |
| Leaves | 29.6 |

Summary: According to the HPLC elution conditions, the polarity distribution of the subject matter (flavonoids) is located in the relatively high water-soluble (high polarity) region and the high oil-soluble (low-polarity) region. Consequently, to completely extract flavonoids, it should be operated in at least four steps (2 times for aqueous phase and 2 times for organic phase), thus making it difficult to commercialize.

The Present Embodiment

Objective: The present invention is aimed at commercial extraction and mass preparation. By selecting extractant with appropriate polarity for extraction, the target component can be precisely qualified and quantified, the extractions steps are therefore being simplified and the greatest amount of target components can be given. Thus, commercialization can therefore be achieved.

C. morifolium source: Daxi Dist., Taoyuan City, Taiwan (R.O.C.)

Extraction condition: (1) Fresh C. morifolium stems and leaves are dried by cold air drying. (2) The flowers were removed from the dried plant and the leaves, thereto obtain a stem sample, a leaf sample, and a stem/leaf sample in which the stems and the leaves were remained with a mass ratio of 7:3. (3) By using an extractant (50% edible alcohol) of 10 times weight of the dried plant to extract the dried plant twice at 60° C. (30 minutes for each time), the extracted product may contain an extraction liquid and an extraction solid, which were subsequently separated from each other. (4) The extraction liquid was concentrated under reduced pressure to a volume of ¼ to obtain an organic phase extract.

Qualitative evaluation method: Chromatographic analysis is used to analyze the organic phase extraction at 415 nm. Detail descriptions can be referred to the above comparative embodiment, and the analysis condition is the same as in Table 2.

Quantitative evaluation method: The spectrophotometer uses colorimetry and quercetin as standards to quantify the total flavonoid content.

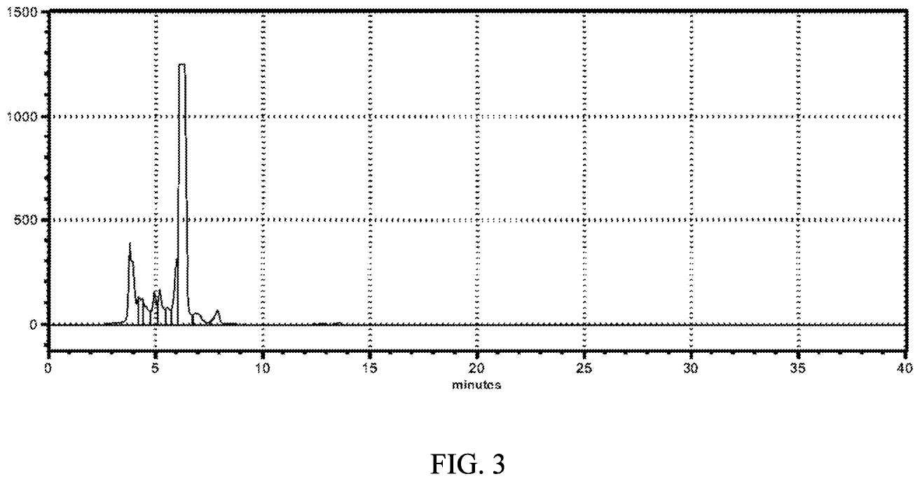

Qualitative results: shown in FIG. 3.

Quantitative results: shown in Table 4.

TABLE 4

| | Flavonoid |
|---|---|
| Portion | (mg/g of dried plant) |
| Stems | 10.4 |
| Leaves | 8.3 |
| Stems/Leaves | 21.0 |

Summary: Qualitative results: Comparing FIGS. 1, 2 of the comparative embodiment to FIG. 3 of the present embedment, it is demonstrated that their fingerprints are similar, indicating that: (1) even the sources of the C. morifolium are different, they may still include similar content. (2) The extraction method proposed by the present invention can give the same extraction result with sacrificing some recovering rate in comparing to convention extraction techniques that uses four extraction steps (two for aqueous phase and two for organic phase), thus making the present invention achievable to the commercial production level. Quantitative results: Using the comparative examples as the reference, the recovery rate (extracted flavonoid/total flavonoid in original strain * 100%) of the stems, the leaves, the stems/leaves are higher than those in conventional commercial extraction techniques (about 40%˜50%).

Animal test: Evaluate the health effects of relieving or alleviating dry eye syndrome with mouse test.

In the foregoing embodiment, three obtained extracts (stems, leaves, stem/leaves) were tested to evaluate their effect of relieving dry eye syndrome by animal models.

Evaluation items: (1) the quality of tear fluid (2) the effect of preventing ultraviolet damage.

Evaluation method: The mice were fed with extracts of leaves, stems, and stem/leaves of C. morifolium for 10 days, and then irradiated with ultraviolet light for 7 consecutive days to cause corneal injury, which will result in dry eye syndrome. The flavonoids in extracts of the leaves and the stem/leaves were both 0.3% (w/w), while in the extracts of stems was 0.05% (w/w). The blank group did not feed with extracts of C. morifolium, and the control group was treated with artificial tears.

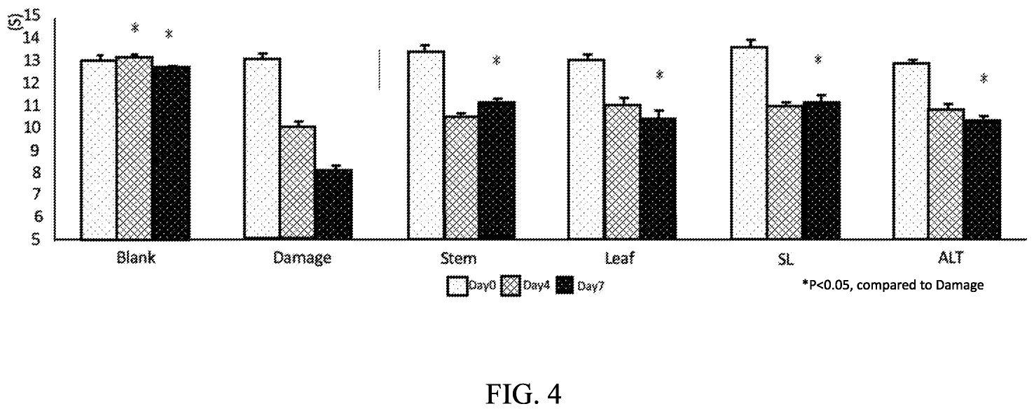

Tear quality results: Please refer to FIG. 4. FIG. 4 is a comparison chart of the quality of tear fluid after irradiating UV to mice and then feeding the mice with the extracts of C. morifolium, wherein the vertical axis is time (unit: second). The first group (Blank) is the blank test (no UV damage group), the second group (Damage) is the UV damage group, the third group (Stem) is UV damage+stems extract administrating group, the fourth group (Leaf) is UV damage+leaves extract administrating group, the fifth group (SL) is UV damage+stems/leaves extract administrating group, and the sixth group (AFT) is UV damage+artificial tear treating group. The tear quality of this experiment refers to the “tear film rupture time.” In normal eyes, the tear film ruptures and becomes tears, but if the eyes cannot secrete tears in time, it will cause dry eyes. Therefore, the longer the tear film maintains, the tear itself not easy to break, representing a good quality. As shown in FIG. 4, the third group Stem (UV damage+stems extract administrating), the fourth group Leaf (UV damage+leaves extract administrating) and the fifth group SL (UV damage+stems/leaves extract administrating) are shown to extend the tear film breakup time and thus is able to shorten the time of dry eye.

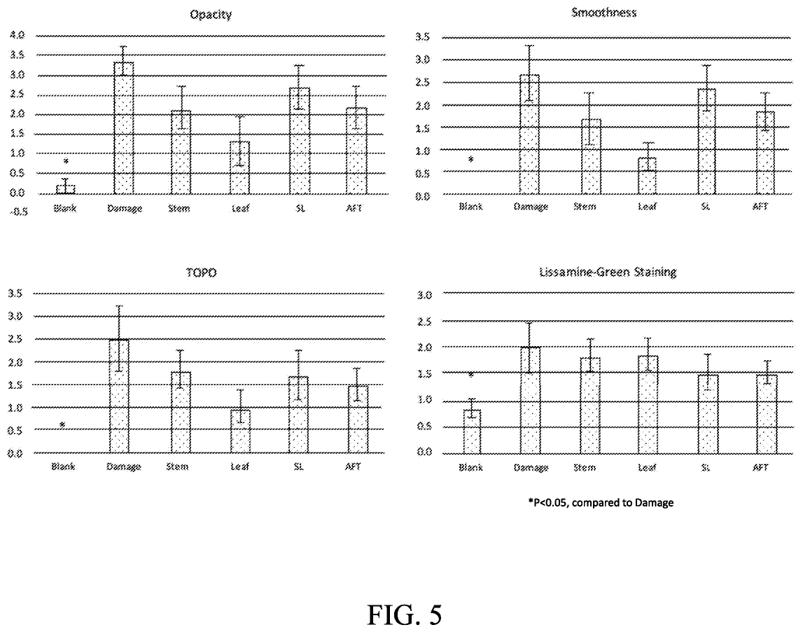

Prevent UV damage: Please refer to FIG. 5, which shows degrees of cornea damage after irradiating ultraviolet light to mice after feeding the mice with the extracts of C. morifolium, and the four sub-pictures respectively represent cornea opacity, cornea smoothness, corneal topography and Lissamine green stain examination. The larger the quantitative number of the degrees, the more serious the degree is. As shown in FIG. 5, the third group Stem (UV damage+stems extract administrating), the fourth group Leaf (UV damage+leaves extract administrating) and the fifth group SL (UV damage+stems/leaves extract administrating), comparing to the second group Damage (UV damage), can effectively reduce damage to the cornea.

The extracts of the present invention can exhibit the benefit of relieving or alleviate the dry eyes (dry eye syndrome), which can be proven by feeding mice with the extracts of C. morifolium.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of extracting flavonoid from Chrysanthemum morifolium, comprising:
providing a dried plant material derived from Chrysanthemum morifolium; and
performing an extraction step with an extractant to obtain an extract solid and an extraction liquid, wherein the extraction liquid comprises flavonoids with a concentration of 8˜22 mg/g of the dried plant material.

2. The method of extracting flavonoid from Chrysanthemum morifolium according to claim 1, wherein the dried plant material of Chrysanthemum morifolium comprises stems and leaves.

3. The method of extracting flavonoid from Chrysanthemum morifolium according to claim 1, wherein the dried plant material of Chrysanthemum morifolium comprises stems, leaves and flowers.

4. The method of extracting flavonoid from Chrysanthemum morifolium according to claim 1, wherein the extractant comprises 40˜60% ethanol.

5. The method of extracting flavonoid from Chrysanthemum morifolium according to claim 2, further comprising an adjusting procedure to render a dry weight ratio of the stems to the leaves become 3:2˜4:1.

6. The method of extracting flavonoid from Chrysanthemum morifolium according to claim 3, further comprising an adjusting procedure to render a dry weight ratio of the stems to the leaves become 3:2˜4:1.

7. The method of extracting flavonoid from Chrysanthemum morifolium according to claim 1, wherein the extraction step is repeated less than 4 times.

8. A method of manufacturing a medicine composition with effect of alleviating dry eye syndrome, comprising:
providing a dried plant material derived from Chrysanthemum morifolium; and
performing an extraction step with an extractant to obtain an extract solid and an extraction liquid, wherein the extraction liquid comprises flavonoids with a concentration of 8˜22 mg/g of the dried plant material.

9. The method of manufacturing a medicine composition with effect of alleviating dry eye syndrome according to claim 8, wherein the dried plant material of Chrysanthemum morifolium comprises stems and leaves.

10. The method of manufacturing a medicine composition with effect of alleviating dry eye syndrome according to claim 8, wherein the dried plant material of Chrysanthemum morifolium comprises stems, leaves and flowers.

11. The method of manufacturing a medicine composition with effect of alleviating dry eye syndrome according to claim 8, wherein the extractant comprises 40˜60% ethanol.

12. The method of manufacturing a medicine composition with effect of alleviating dry eye syndrome according to claim 9, further comprising an adjusting step to render a dry weight ratio of the stems to the leaves become 3:2˜4:1.

13. The method of manufacturing a medicine composition with effect of alleviating dry eye syndrome according to claim 10, further comprising an adjusting step to render a dry weight ratio of the stems to the leaves become 3:2˜4:1.

14. The method of manufacturing a medicine composition with effect of alleviating dry eye syndrome according to claim 8, wherein the medicine composition comprises the flavonoids with a concentration of 0.05%˜0.3% (w/w).
